(19) United States
(12) Patent Application Publication
(10) Pub. No.: US 2022/0192542 A1
(43) Pub. Date: Jun. 23, 2022

(54) MICRONEEDLE PATCH, REALTIME BLOOD SUGAR MONITORING DEVICE, AND REALTIME BLOOD SUGAR MONITORING METHOD USING SAME

(71) Applicant: Philosys Co., Ltd., Gunsan-si, Jeollabuk-do (KR)

(72) Inventors: Jong Jin KIM, Suwon-si, Gyeonggi-do (KR); Jae Hyun HWANG, Gwangju-si, Gyeonggi-do (KR)

(73) Assignee: Philosys Co., Ltd., Gunsan-si, Jeollabuk-do (KR)

(21) Appl. No.: 17/441,836

(22) PCT Filed: Mar. 26, 2020
(86) PCT No.: PCT/KR2020/004098

§ 371 Date: Sep. 22, 2021

(30) Foreign Application Priority Data
Mar. 27, 2019 (KR) 10-2019-0035021
Mar. 27, 2019 (KR) 10-2019-0035022

(51) Int. Cl. A61B 5/145; A61B 5/1473; A61B 5/00

(52) U.S. Cl. CPC A61B 5/14532; A61B 2560/0412; A61B 5/685; A61B 5/14735

(57) ABSTRACT

Disclosed are a microneedle patch, a realtime blood sugar monitoring device, and a realtime blood sugar monitoring method using the realtime blood sugar monitoring device. The realtime blood sugar monitoring device causes a different color to be reversibly expressed according to the concentration of sugar included in the skin of a human body, and, thus, if the device is utilized, the concentration of the sugar in the human body can be measured in realtime in a minimally invasive manner. Also, the present invention shows the measured concentration of the sugar by connecting to a wearable device and, thereby, allows a user to more conveniently check changes in the concentration of the sugar in realtime.

### TECHNICAL FIELD

Example embodiments relate to a microneedle patch, a realtime blood sugar monitoring device, and a realtime blood sugar monitoring method using the realtime blood sugar monitoring device.

### BACKGROUND ART

In a current medical system, health management for patients may be performed as a patient himself/herself visits a medical institution or center and receives medical checkups and physical condition measurements. In a social structure that is changing worldwide, for example, in an aging society where a population aged 65 and over occupies more than 20% of the total population, it is expected that an elderly population that needs to be managed by each nation increases and a national medical support cost increases. In Korea, an aging population is increasing rapidly. Thus, a future medical system is expected to focus on preventive approaches, such as continuous bio-monitoring of adult diseases such as hypertension, diabetes, and heart disease, instead of diagnosis or treatment, using U-health care (or remote health management) which is based on information technology (IT) innovations and infrastructure expansion.

For example, blood sugar may be measured by an electrochemical method. When a blood sample obtained by collecting blood from a user is applied to a specimen having a chemical reaction, sugar in the blood is oxidized by glucose oxidase, and the glucose oxidase is reduced. In addition, an electron acceptor oxidizes the glucose oxidase and the electron acceptor itself is reduced. The reduced electron acceptor loses electrons on an electrode surface to which a constant voltage is applied and is again electrochemically oxidized. The concentration of the sugar (or glucose) in the blood sample is proportional to the amount of current generated in the process in which the electron acceptor is oxidized, and thus the concentration of the blood sugar may be measured by measuring this amount of current.

On the other hand, a needle may be used to obtain a sample from a living body, detect biometric information of a user, or inject a drug into a living body. As the needle, a microneedle having a diameter of millimeters (mm) is used in most cases.

For example, to measure blood sugar (which refers to a glucose level in the blood) of diabetics, a level of glucose in the blood of a measurement target (e.g., a diabetic) may be measured several times a day, for example, after waking up and before and after a meal, by collecting the blood using a blood sugar measuring device such as a blood sugar strip. However, such a blood sugar measuring device may collect the blood from a finger of the measurement target using a blood collecting needle (e.g., a lancet) each time measuring blood sugar, and measure the blood sugar in the collected blood using a strip sensor and a reader.

Thus, there is a need for a technology for measuring a level of blood sugar in real time. Accordingly, the inventor(s) of the present disclosure has completed the present disclosure by developing a device capable of optically measuring the concentration of sugar based on a change in color during research on a technology for monitoring blood sugar in real time using a microneedle.

In this regard, Korean Patent Registration No. 10-1542549 discloses a microneedle array for biosensing and drug delivery.

### DISCLOSURE OF THE INVENTION

### Technical Goals

To resolve issues described above, an aspect provides a realtime blood sugar monitoring device and a microneedle patch.

Another aspect provides a realtime blood sugar monitoring method using a realtime blood sugar monitoring device.

Additional aspects of example embodiments will be set forth in part in the description which follows and, in part, will be apparent from the description, or may be learned by practice of the disclosure.

### Technical Solutions

One Aspect of the Present Disclosure

According to an aspect, there is provided a realtime blood sugar monitoring device including a sensor including a microneedle configured to invade a skin of a human body, a measurer configured to measure a color generated by the sensor, and a notifier configured to notify a user of the color measured by the measurer. The sensor may express the color by reacting with sugar included in a body fluid in the skin of the human body.

The sensor may reversibly express a different color based on a concentration of the sugar.

The sensor may include a polymer matrix that reversibly expresses the different color based on the concentration of the sugar.

The polymer matrix may be in contact with an upper side of the microneedle.

The sensor may include an enzyme that reacts with the sugar included in the body fluid.

The enzyme may be glucose oxidase.

The sensor may include a polymer matrix in contact with an upper side of the microneedle.

The sensor may include a chromogenic body that reversibly expresses the different color by sensing an electron, hydrogen peroxide, or a change in pH that are generated from the reaction between the sugar included in the body fluid and the enzyme.

The chromogenic body may include a substance selected from a group consisting of a magnetic nanoparticle, a ruthenium complex, a chromogenic indicator, a dye, a para-hydroxyphenyl acetic acid, a lectin, and a combination thereof.

The realtime blood sugar monitoring device may release, to an outside, the body fluid that is already reacted on a periodic basis.

The realtime blood sugar monitoring device may further include a controller configured to control the measurer and the notifier, and a power supply configured to supply needed power to the measurer, the notifier, and the controller.

The realtime blood sugar monitoring device may further include an amplifier configured to amplify a signal measured by the measurer, and an analog-to-digital converter (ADC) configured to digitize a signal amplified by the amplifier.

The notifier may convert the color measured by the measurer into the concentration of the sugar in the body fluid and visually provide the user with a result of the converting through a display.

Another Aspect of the Present Disclosure

According to another aspect, there is provided a realtime blood sugar monitoring method using the realtime blood sugar monitoring device, the realtime blood sugar monitoring method including invading, by the microneedle, a skin of a human body, expressing a color as sugar included in a body fluid in the skin of the human body reacts with the sensor, measuring the expressed color, and notifying a user of the measured color.

According to still another aspect, there is provided a microneedle patch including a microneedle configured to invade a skin of a human body, and a sensor configured to express a color by coming into contact with the microneedle and reacting with sugar included in a body fluid in the skin of the human body.

The sensor may reversibly express a different color based on a concentration of the sugar.

The sensor may include a polymer matrix that reversibly expresses the different color based on the concentration of the sugar.

The polymer matrix may be in contact with an upper side of the microneedle.

The sensor may include an enzyme that reacts with the sugar included in the body fluid.

The enzyme may be glucose oxidase.

The sensor may include a polymer matrix in contact with an upper side of the microneedle.

The sensor may include a chromogenic body that reversibly expresses the different color by sensing an electron, hydrogen peroxide, or a change in pH that are generated from the reaction between the sugar included in the body fluid and the enzyme.

The chromogenic body may include a substance selected from a group consisting of a magnetic nanoparticle, a ruthenium complex, a chromogenic indicator, a dye, a para-hydroxyphenyl acetic acid, a lectin, and a combination thereof.

### Effects

According to example embodiments described herein, a realtime blood sugar monitoring device and a microneedle patch may reversibly express or develop different colors based on a concentration of sugar included in a skin of a human body, and may thus minimally invasively measure the concentration of the sugar in the human body in real time.

In addition, the realtime blood sugar monitoring device and the microneedle patch may display the measured concentration of the sugar in conjunction with a wearable device, thereby allowing a user to verify a realtime change in the concentration of sugar more conveniently.

It should be understood that the effects of the present disclosure are not limited to the effects described above, but are construed as including all effects that can be inferred from the configurations and features described in the following description or claims of the present disclosure.

### BRIEF DESCRIPTION OF DRAWINGS

FIG. 1 is a diagram illustrating an example of a realtime blood sugar monitoring device according to an example embodiment.

FIG. 2 is a detailed diagram illustrating an example of a realtime blood sugar monitoring device according to an example embodiment.

FIG. 3 is a diagram illustrating an example of a realtime blood sugar monitoring device including an enzyme according to an example embodiment.

FIG. 4 is a diagram illustrating an example of a realtime blood sugar monitoring device including a chromogenic body according to an example embodiment.

FIG. 5 is a diagram illustrating another example of a realtime blood sugar monitoring device including an enzyme according to an example embodiment.

FIG. 6 is a diagram illustrating another example of a realtime blood sugar monitoring device including a chromogenic body according to an example embodiment.

FIG. 7 is a diagram illustrating an example of a microneedle patch according to an example embodiment.

FIG. 8 is a detailed diagram illustrating an example of a microneedle patch according to an example embodiment.

FIG. 9 is a diagram illustrating an example of a microneedle patch including an enzyme according to an example embodiment.

FIG. 10 is a diagram illustrating an example of a microneedle patch including a chromogenic body according to an example embodiment.

FIG. 11 is a diagram illustrating another example of a microneedle patch including an enzyme according to an example embodiment.

FIG. 12 is a diagram illustrating another example of a microneedle patch including a chromogenic body according to an example embodiment.

### BEST MODE FOR CARRYING OUT THE INVENTION

Hereinafter, some examples will be described in detail with reference to the accompanying drawings. However, various alterations and modifications may be made to the examples. Here, the examples are not construed as limited to the disclosure and should be understood to include all changes, equivalents, and replacements within the idea and the technical scope of the disclosure.

Although terms such as “first,” “second,” and “third” may be used herein to describe various members, components, regions, layers, or sections, these members, components, regions, layers, or sections are not to be limited by these terms. Rather, these terms are only used to distinguish one member, component, region, layer, or section from another member, component, region, layer, or section. Thus, a first member, component, region, layer, or section referred to in the examples described herein may also be referred to as a second member, component, region, layer, or section without departing from the teachings of the examples.

Throughout the specification, when a component is described as being “connected to” or “coupled to” another component, it may be directly “connected to” or “coupled to” the other component, or there may be one or more other components intervening therebetween. In contrast, when an element is described as being “directly connected to” or “directly coupled to” another element, there can be no other elements intervening therebetween.

The terminology used herein is for the purpose of describing particular examples only and is not to be limiting of the examples. As used herein, the singular forms “a,” “an,” and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which this disclosure pertains based on an understanding of the present disclosure. Terms, such as those defined in commonly used dictionaries, are to be interpreted as having a meaning that is consistent with their meaning in the context of the relevant art and the present disclosure, and are not to be interpreted in an idealized or overly formal sense unless expressly so defined herein.

Also, in the description of example embodiments, detailed description of structures or functions that are thereby known after an understanding of the disclosure of the present application will be omitted when it is deemed that such description will cause ambiguous interpretation of the example embodiments. Hereinafter, examples will be described in detail with reference to the accompanying drawings, and like reference numerals in the drawings refer to like elements throughout.

One Aspect of the Present Disclosure

In one aspect, there is provided a realtime blood sugar monitoring device 101 may include a sensor 200 including a microneedle 220 configured to invade a skin 110 of a human body, a measurer 300 configured to measure a color generated by the sensor 200, and a notifier 400 configured to notify a user of the color measured by the measurer 300. The sensor 200 may express or develop the color by reacting with sugar included in a body fluid in the skin 110 of the human body.

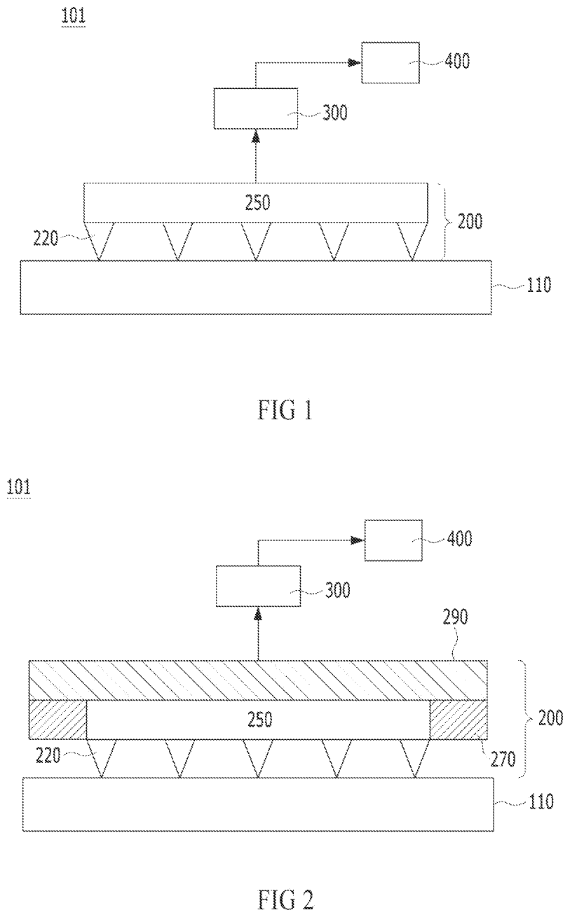

Hereinafter, the realtime blood sugar monitoring device 101 will be described in detail with reference to FIGS. 1 through 6 according to one aspect of the present disclosure. FIG. 1 is a diagram illustrating the realtime blood sugar monitoring device 101, and FIGS. 2 through 6 are detailed diagrams illustrating the realtime blood sugar monitoring device 101 according to example embodiments.

According to an example embodiment, the realtime blood sugar monitoring device 101 may include the sensor 200, and the sensor 200 may react with sugar included in a body fluid in the skin 110 of the human body and reversibly express or develop a different color based on a concentration of the sugar. The sugar in the body fluid may be connected to the sensor 200 through the microneedle 220. The microneedle 220 may have a non-limiting number of needles, be 500±200 micrometers (μm) in size to such an extent that the user does not feel pain therefrom, be low in production cost, and be formed of a medical material that is harmless to the human body.

According to an example embodiment, the realtime blood sugar monitoring device 101 may further include an adhesive portion 270 and a film 290 as illustrated in FIG. 2. The adhesive portion 270 may be provided for effective adhesion of the realtime blood sugar monitoring device 101 to the skin 110 of the human body, and provided in any shape that enables the adhesion to the skin 110. However, it may be desirably disposed at both ends of a polymer matrix 250. In addition, the adhesive portion 270 may be formed of any material that is well attached to the skin 110 or other known adhesive materials. However, it may be desirably formed of an adhesive material that is harmless to the skin 110 of the human body. The film 290 may be included to stably fix the shape of the polymer matrix 250, and be disposed between the polymer matrix 250 and the measurer 300. In this case, the film 290 may be desirably transparent such that the measurer 300 effectively measures a color expressed or developed in the polymer matrix 250, and be desirably waterproof because it is likely to be exposed to the body fluid. In addition, the film 290 may be desirably formed of a material that does not react to the sugar, or electrons, hydrogen peroxide, or a pH change that are generated by a reaction between the sugar and an enzyme 230. That is, the film 290 may be desirably a transparent waterproof film that does not react to the sugar, or the electrons, the hydrogen peroxide, or the pH change that are generated by the reaction between the sugar and the enzyme 230.

According to an example embodiment, the realtime blood sugar monitoring device 101 may release, to an outside, the body fluid that is already reacted on a periodic basis. The body fluid may react in an area of the microneedle 220 and then be absorbed back into the human body, or may be extracted up to the polymer matrix 250 through the microneedle 220 to react therein. That is, the body fluid reacted in the polymer matrix 250 may stay in the polymer matrix 250 without being absorbed again into the human body. In this case, to measure a concentration of sugar included in the body fluid after a certain period elapses, the already reacted body fluid may need to be released to the outside. To this end, the sensor 200 may be provided in a form having one side thereof being slightly opened to the outside. Thus, the body fluid may be released to the outside when the user presses the sensor 200, or released to the outside through a pump.

According to an example embodiment, the realtime blood sugar monitoring device 101 may further include a controller configured to control the measurer 300 and the notifier 400, and a power supply configured to supply needed power to the measurer 300, the notifier 400, and the controller.

According to an example embodiment, the realtime blood sugar monitoring device 101 may have a minimized size using a micro-electro-mechanical systems (MEMS) technology, and be configured as an active device using, as the power supply, a general battery, an ultrasmall charge and discharge battery, or an ultrasmall supercapacitor. In addition, in the case of a manual type of which a communicator of the realtime blood sugar monitoring device 101 uses low-pass (LC) resonance formed with an inductor and a capacitor, an antenna capable of wirelessly communicating with the realtime blood sugar monitoring device 101 of the manual type may need to be included in a communication terminal such as a mobile phone or a smartphone. When using the realtime blood sugar monitoring device 101 of the manual type, the wireless communication between the realtime blood sugar monitoring device 101 and the communication terminal may be performed by a magnetic induction-based coupling method. That is, using a principle of supplying power to the realtime blood sugar monitoring device 101 using an electromotive force generated from the antenna of such an external terminal, it is possible to configure it as a circuit that does not have a separate power supply.

According to an example embodiment, the power supply may be used to operate a microcontroller, and supply power to the measurer 300 that requires a separate power supply. In this case, the microcontroller may store therein a separate measurement process in a memory, and the measurement process may be performed according to a procedure programmed individually according to a type of data to be measured. An initial state may be a standby state in which power is not supplied to the microcontroller. When a wake-up signal is received from an external device, a measurement may be started in the standby state.

According to an example embodiment, the realtime blood sugar monitoring device 101 may further include an amplifier configured to amplify a signal measured by the measurer 300 and an analog-to-digital converter (ADC) configured to digitize a signal amplified by the amplifier.

The signal measured by the measurer 300 may be transmitted to the notifier 400 through the communicator, and the notifier 400 may be, for example, a mobile phone or a smartphone. The measured signal may be transmitted and received using a technology for wireless communication between the measurer 300 and the mobile phone or smartphone. Here, in the case of a manual type of which the communicator uses LC resonance formed with an inductor and a capacitor, an antenna capable of wirelessly communicating with the measurer 300 of the manual type may need to be embedded in the measurer 300 and a communication terminal such as a mobile phone or a smartphone. When using the measurer 300 of the manual type, the wireless communication between the measurer 300 and the communication terminal may be performed by a magnetic induction-based coupling method. When transmitting and receiving data using the magnetic induction-based coupling method, the terminal may need to be disposed within an appropriate distance with the terminal and the measurer 300 being disposed on a straight line. In addition, it is possible to transmit and receive data wirelessly by embedding a separate communication module for Zigbee communication and Bluetooth communication in the terminal and the measurer 300 as a pair, or establish the wireless communication by providing a separate communication module outside the terminal. In this case, using the wireless communication module, it is possible to transmit and receive data within 10±5 meters (m) regardless of positions of the measurer 300 and the terminal.

According to an example embodiment, all methods of measuring blood sugar using the realtime blood sugar monitoring device 101 may be started by a content of a mobile phone or a terminal, or an application of a smartphone. The content or application may be individually configured to interwork with a certain device, for example, the realtime blood sugar monitoring device 101, and may also be configured as an integrated content or application capable of interworking with all devices including, for example, the realtime blood sugar monitoring device 101. When, in the content and application, a type of device for measurement, for example, the realtime blood sugar monitoring device 101, is selected and personal information is input, and a start button is then pressed, the content or application may transmit a wake-up signal to the device 101. By the wake-up signal, the measurement by the device 101 may be started. The content or application may display a current state of the user by comparing, to existing reference data, a data value received through the communicator and other result values. In this case, when a health condition of the user is out of a normal range, for example, when the user has an abnormal concentration of sugar in the body, the content or application may provide the user with a simple health management method and provide, at the same time, information for the user to transmit related data to his/her guardian and doctor. However, when a signal is weak due to a poor attachment between a portion of the user being measured and the device 101, or when a signal is weak due to a poor contact between the device 101 and the terminal, the content or application may transmit such a problem to the user such that the measurement is performed smoothly.

According to an example embodiment, the notifier 400 may convert a color measured by the measurer 300 into a concentration of sugar in a body fluid and visually provide the concentration to the user through a display.

According to an example embodiment, the sensor 200 may include the polymer matrix 250 that reversibly expresses or develops a different color based on a concentration of sugar, and/or a chromogenic body 260 that reversibly expresses or develops a different color by sensing electrons, hydrogen peroxide, or a change in pH that are generated by a reaction between the sugar and the enzyme 230.

Hereinafter, an example where the polymer matrix 250 that reversibly expresses a different color based on a concentration of sugar is included, and an example where the chromogenic body 260 that reversibly expresses a different color by sensing electrons, hydrogen peroxide, or a change in pH that are generated by a reaction between the sugar and the enzyme 230 will be described, respectively.

The example where the realtime blood sugar monitoring device 101 includes the polymer matrix 250 that reversibly expresses a different color based on a concentration of sugar will be first described hereinafter with reference to FIG. 1.

The polymer matrix 250 may be in contact with an upper side of the microneedle 220. That is, a body fluid in the skin 110 of the human body may be extracted into the polymer matrix 250 through the microneedle 220, and sugar included in the extracted body fluid may directly react with the polymer matrix 250 and a color of the polymer matrix 250 may thereby be changed according to a concentration of the sugar.

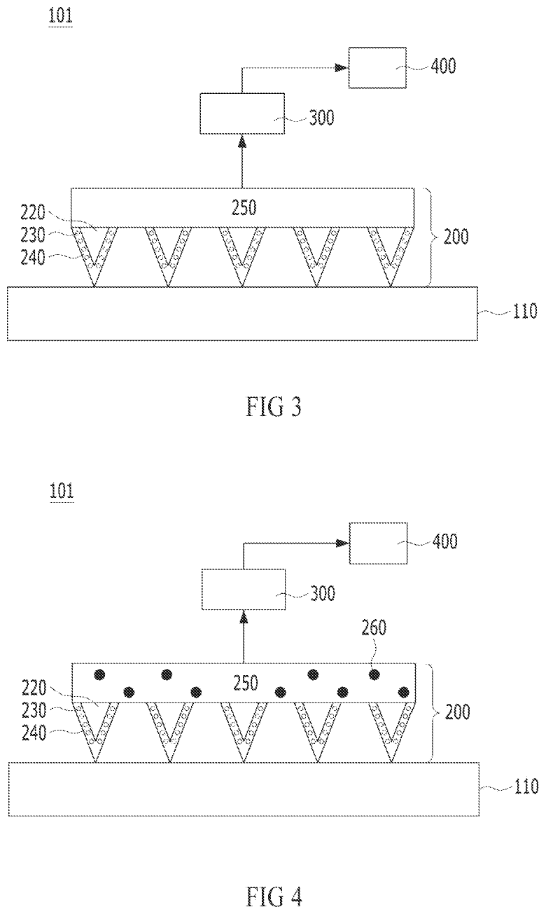

The example where the realtime blood sugar monitoring device 101 includes the chromogenic body 260 that reversibly expresses a different color by sensing electrons, hydrogen peroxide, or a change in pH that are generated by a reaction between the sugar and the enzyme 230 will be described hereinafter with reference to FIGS. 3 through 6.

The sensor 200 may include the enzyme 230 that reacts with sugar included in a body fluid, and the enzyme 230 may be desirably glucose oxidase (GOx). The sugar described herein may also be referred to as glucose. In this case, the glucose oxidase (GOx) may react with the sugar, and the reaction may be represented by Reaction Equation 1 below.

GOx+glucose→reduced GOx+gluconolactone Reduced GOx+O₂→GOx+H₂O₂ H₂O₂→O₂+2H⁺+2e⁻ [Reaction Equation 1]

That is, as represented by Reaction Equation 1 above, the glucose oxidase may react with the sugar (or glucose) to generate hydrogen peroxide ((H₂O₂) and/or electrons (e⁻). In this case, the glucose oxidase is oxidized again after being reduced, and may thus react reversibly with the sugar, enabling a continuous reaction. In addition, a pH change may occur due to the generated hydrogen peroxide. Thus, as the enzyme 230 reacts with the sugar in the body fluid, the electrons, hydrogen peroxide, or pH change may be represented thereby.

According to an example embodiment, the sensor 200 may include the chromogenic body 260 that reversibly expresses a different color by sensing the electrons, hydrogen peroxide, or pH change that are generated by the reaction between the sugar included in the body fluid and the enzyme 230.

The chromogenic body 260 may include a substance selected from a group consisting of a magnetic nanoparticle, a ruthenium complex, a chromogenic indicator, a dye, a para-hydroxyphenyl acetic acid, a lectin, and combinations thereof.

The magnetic nanoparticle may be peroxidase-active, and may include a substance selected from a group consisting of iron oxide, ferrite, alloy, and combinations thereof. In detail, the iron oxide may be, for example, Fe₂O₃ or Fe₃O₄. The ferrite may be, for example, CoFe₂O₄ or MnFe₂O₄. The alloy may be, for example, FePt or CoPt. However, the magnetic nanoparticle may not directly generate a color change by reacting with hydrogen peroxide, but generate the color change through a chromogenic substrate. In this case, the chromogenic substrate may include a substance selected from a group consisting of Amplex Red, ABTS, TMB, o-phenylenediamine dihydrochloride (OPD), 3,3′-diaminobenzidine (DAB), and combinations thereof. The magnetic nanoparticle may be effectively used as the chromogenic body 260 that reversibly expresses the color because it is effectively separable and reusable using a magnetic force.

The ruthenium complex may be ruthenium II complex (Ru(ddp)₃²), and used to measure a concentration of sugar using its characteristic that its fluorescence intensity decreases because oxygen consumption increases as the concentration of the sugar increases.

The chromogenic indicator may react with the generated hydrogen peroxide in the presence of peroxidase to generate a different color or generate chemiluminescence, and may include, for example, 3-hydroxy-2,4,6-triiodide benzoic acid or 3-hydroxy-2,4,6-tribromobenzoic acid.

The dye may be, for example, a xanthene-type dye or a fluorescein-type dye, and may generate an optical signal that changes dependently on a concentration based on the concentration of sugar.

The para-hydroxyphenyl acetic acid may exhibit strong fluorescence in proportion to a concentration of sugar, and additionally use a ruthenium porphyrin complex (RuO₂) as a catalyst for decomposing a dimer to achieve a reversible reaction.

The lectin may be a sugar-binding lectin, for example, Con A or glucose oxidase.

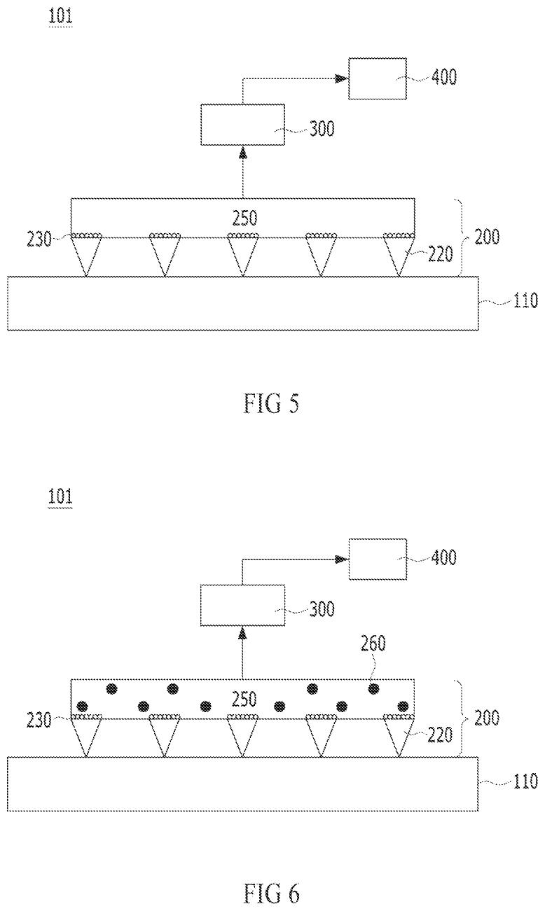

According to an example embodiment, the sensor 200 may include the polymer matrix 250 that is in contact with an upper side of the microneedle 220, and the chromogenic body 260 may be dispersed in the polymer matrix 250. In this case, the enzyme 230 may be dispersed in the microneedle 220 (refer to FIGS. 3 and 4) or be dispersed in the polymer matrix 250 (refer to FIGS. 5 and 6).

According to an example embodiment, when the enzyme 230 is dispersed in the microneedle 220, the microneedle 220 may further include a coating layer 240 on an outer side thereof. In this case, the coating layer 240 may be of a porous structure including a plurality of pores through which other components of relatively large size included in the body fluid may not pass, but sugar of relatively small size may pass. In this case, the sugar included in the body fluid may penetrate the coating layer 240 and react with the enzyme 230 dispersed in the microneedle 220 to generate electrons or hydrogen peroxide, or change pH. The generated electrons or hydrogen peroxide or the change in pH occurring thereby may react with the chromogenic body 260 dispersed in the polymer matrix 250 to express or develop a color.

That the enzyme 230 is dispersed in the polymer matrix 250 may be desirably a case in which the enzyme 230 is dispersed in a portion in contact with the microneedle 220 in the polymer matrix 250. In this case, sugar included in the body fluid may be extracted into the polymer matrix 250 through the microneedle 220, and the extracted sugar may react with the enzyme 230 dispersed in the polymer matrix 250. Thus, the electrons, hydrogen peroxide, or the pH change be generated or occur thereby. Thus, the generated electrons, hydrogen peroxide, or pH change may react with the chromogenic body 260 dispersed in the polymer matrix 250 to express or develop a color.

Another Aspect of the Present Disclosure

In another aspect, there is provided a realtime blood sugar monitoring method using the realtime blood sugar monitoring device 101 according to the aspect described above may include invading, by the microneedle 220, a skin 110 of the human body, expressing a color as sugar included in a body fluid in the skin 110 of the human body reacts with the sensor 200, measuring the expressed color, and notifying a user of the measured color.

Although a description considered redundant as it is already provided in accordance with the foregoing aspect will be omitted hereinafter for brevity, the description is also applied the same as described above to the other aspect to be described hereinafter.

Hereinafter, the realtime blood sugar monitoring method according to the other aspect will be described in detail.

According to an example embodiment, the realtime blood sugar monitoring method may include allowing the microneedle 220 to invade the skin 110 of the human body.

The microneedle 220 may have a non-limiting number of needles, be 500±200 μm in size to such an extent that a user does not feel pain therefrom, be low in production cost, and be formed of a medical material that is harmless to the human body.

According to an example embodiment, the realtime blood sugar monitoring method may include expressing a color by allowing the sensor 200 to react with the sugar in the body fluid in the skin 110 of the human body.

According to an example embodiment, the sensor 200 may include the polymer matrix 250 that reversibly expresses a different color based on a concentration of sugar, and/or the chromogenic body 260 that reversibly expresses a different color by sensing electrons, hydrogen peroxide, a change in pH that are generated by a reaction between the sugar and the enzyme 230.

According to an example embodiment, the realtime blood sugar monitoring method may include measuring the expressed color and notifying the user of the measured color.

According to an example embodiment, the realtime blood sugar monitoring device 101 may further include a controller configured to control the measurer 300 and the notifier 400, and a power supply configured to supply needed power to the measurer 300, the notifier 400, and the controller.

According to an example embodiment, the realtime blood sugar monitoring device 101 may further include a controller configured to control the measurer 300 and the notifier 400, and a power supply configured to supply needed power to the measurer 300, the notifier 400, and the controller.

According to an example embodiment, the realtime blood sugar monitoring device 101 may have a minimized size through a MEMS technology, and be configured as an active device using, as the power supply, a general battery, an ultrasmall charge and discharge battery, or an ultrasmall supercapacitor. In addition, in the case of a manual type of which a communicator of the realtime blood sugar monitoring device 101 uses LC resonance formed with an inductor and a capacitor, an antenna capable of wirelessly communicating with the realtime blood sugar monitoring device 101 of the manual type may need to be included in a communication terminal such as a mobile phone or a smartphone. When using the realtime blood sugar monitoring device 101 of the manual type, the wireless communication between the realtime blood sugar monitoring device 101 and the communication terminal may be performed by a magnetic induction-based coupling method. That is, using a principle of supplying power to the realtime blood sugar monitoring device 101 using an electromotive force generated from the antenna of such an external terminal, it is possible to configure it as a circuit that does not have a separate power supply.

According to an example embodiment, the power supply may be used to operate a microcontroller, and supply power to the measurer 300 that requires a separate power supply. In this case, the microcontroller may store a separate measurement process in a memory, and the measurement process may be performed according to a procedure programmed individually according to a type of data to be measured. An initial state may be a standby state in which power is not supplied to the microcontroller. When a wake-up signal is received from an external device, a measurement may be started in the standby state.

According to an example embodiment, the realtime blood sugar monitoring device 101 may further include an amplifier configured to amplify a signal measured by the measurer 300 and an ADC configured to digitize a signal amplified by the amplifier.

The signal measured by the measurer 300 may be transmitted to the notifier 400 through the communicator, and the notifier 400 may be, for example, a mobile phone or a smartphone. The measured signal may be transmitted and received through a technology for wireless communication between the measurer 300 and the mobile phone or smartphone. Here, in the case of a manual type of which the communicator uses LC resonance formed with an inductor and a capacitor, an antenna capable of wirelessly communicating with the measurer 300 of the manual type may need to be embedded in the measurer 300 and a communication terminal such as a mobile phone or a smartphone. When using the measurer 300 of the manual type, the wireless communication between the measurer 300 and the communication terminal may be performed by a magnetic induction-based coupling method. When transmitting and receiving data using the magnetic induction-based coupling method, the terminal may need to be disposed within an appropriate distance with the terminal and the measurer 300 being disposed on a straight line. In addition, it is possible to transmit and receive data wirelessly by embedding a separate communication module for Zigbee communication and Bluetooth communication in the terminal and the measurer 300 as a pair, and establish the wireless communication by providing a separate communication module outside the terminal. In this case, using the wireless communication module, it is possible to transmit and receive data within 10±5 m regardless of positions of the measurer 300 and the terminal.

According to an example embodiment, all methods of measuring blood sugar using the realtime blood sugar monitoring device 101 may be started by a content of a mobile phone or a terminal, or an application of a smartphone. The content or application may be individually configured to interwork with a certain device, for example, the realtime blood sugar monitoring device 101, and may also be configured as an integrated content or application capable of interworking with all devices including, for example, the realtime blood sugar monitoring device 101. When, in the content and application, a type of device for measurement, for example, the realtime blood sugar monitoring device 101, is selected and personal information is input, and a start button is then pressed, the content or application may transmit a wake-up signal to the device 101. By the wake-up signal, the measurement by the device 101 may be started. The content or application may display a current state of the user by comparing, to existing reference data, a data value received through the communicator and other result values. In this case, when a health condition of the user is out of a normal range, for example, when the user has an abnormal concentration of sugar in the body, the content or application may provide the user with a simple health management method and provide, at the same time, information for the user to transmit related data to his/her guardian and doctor. However, when a signal is weak due to a poor attachment between a portion of the user being measured and the device 101, or when a signal is weak due to a poor contact between the device 101 and the terminal, the content or application may transmit such a problem to the user such that the measurement is performed smoothly.

According to an example embodiment, the notifier 400 may visually provide the user with the color measured by the measurer 300 through a display.

According to an example embodiment, the realtime blood sugar monitoring method may further include discharging or releasing, to an outside, the body fluid that is already reacted on a periodic basis.

The body fluid may react in an area of the microneedle 220 and then be absorbed back into the human body, or may be extracted into the polymer matrix 250 through the microneedle 220 to be reacted therein. That is, the body fluid reacted in the polymer matrix 250 may stay in the polymer matrix 250 without being absorbed again into the human body. In this case, to measure a concentration of sugar included in the body fluid after a certain period, the body fluid that is already reacted may need to be released to the outside. To this end, the sensor 200 may be in a form having one side slightly opened to the outside, and the user may release the body fluid to the outside by pressing the sensor 200, or release the body fluid to the outside using a pump.

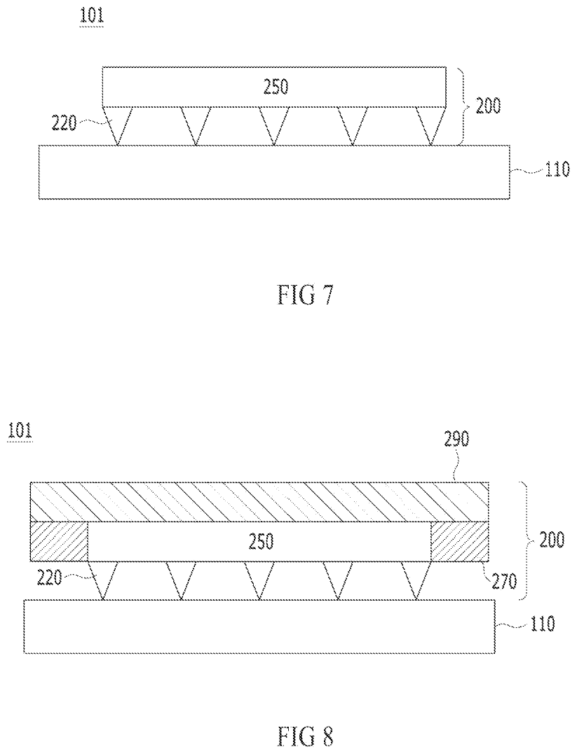

Hereinafter, an example where the realtime blood sugar monitoring device 101 is implemented as a microneedle patch 101 will be described with reference to FIGS. 7 through 12.

Still Another Aspect of the Present Disclosure

In still another aspect, there is provided a microneedle patch 101 including a microneedle 220 configured to invade a skin 110 of a human body, and a sensor 200 configured to express a color by coming into contact with the microneedle 220 and reacting with sugar included in a body fluid in the skin 110 of the human body.

Hereinafter, the microneedle patch 101 according to still another aspect will be described in detail with reference to FIGS. 7 through 12. FIG. 7 is a diagram illustrating the microneedle patch 101, and FIGS. 8 through 12 are diagrams illustrating detailed examples of the microneedle patch 101.

According to an example embodiment, the microneedle patch 101 may include the sensor 200, and the sensor 200 may react with the sugar included in the body fluid in the skin 110 of the human body and reversibly express a different color based on a concentration of the sugar. In this case, the sugar included in the body fluid may be connected to the sensor 200 through the microneedle 220. The microneedle 220 may have a non-limiting number of needles, and be 500±200 μm in size to such an extent that a user does not feel pain therefrom, be low in production cost, and be formed of a medical material that is harmless to the human body.

According to an example embodiment, the microneedle patch 101 may further include an adhesive portion 270 and a film 290 as illustrated in FIG. 8. The adhesive portion 270 may be provided for effective adhesion of the microneedle patch 101 to the skin 110 of the human body, and provided in any shape that enables the adhesion to the skin 110. However, it may be desirably disposed at both ends of a polymer matrix 250. In addition, the adhesive portion 270 may be formed of any material that is well attached to the skin 110 or other known adhesive materials. However, it may be desirably formed with an adhesive material that is harmless to the skin 110 of the human body. The film 290 may be included to stably fix the shape of the polymer matrix 250, and be disposed on the polymer matrix 250. In this case, the film 290 may be desirably transparent such that a color expressed or developed in the polymer matrix 250 is effectively measured from outside, and be desirably waterproof because it is likely to be exposed to the body fluid. In addition, the film 290 may be desirably formed of a material that does not react to the sugar, or electrons, hydrogen peroxide, or a change in pH that are generated by a reaction between the sugar and an enzyme 230. That is, the film 290 may be desirably a transparent waterproof film that does not react to the sugar, or the electrons, hydrogen peroxide, or change in pH that are generated by the reaction between the sugar and the enzyme 230.

According to an example embodiment, the microneedle patch 101 may release, to an outside, the body fluid that is already reacted on a periodic basis. The body fluid may react in an area of the microneedle 220 and then be absorbed back into the human body, or may be extracted into the polymer matrix 250 through the microneedle 220 to be reacted therein. That is, the body fluid reacted in the polymer matrix 250 may stay in the polymer matrix 250 without being absorbed again in the human body. In this case, to measure a concentration of sugar included in the body fluid after a certain period, the reacted body fluid may need to be released to the outside. To this end, the sensor 200 may be provided in a form having one side thereof being slightly opened to the outside. Thus, the body fluid may be released to the outside when the user presses the sensor 200, or released to the outside through a pump.

According to an example embodiment, the microneedle patch 101 may further include a measurer and a notifier. The measurer may measure the color expressed by the sensor 200, and the notifier may notify the user of the measured color. In this case, the microneedle patch 101 may further include a controller configured to control the measurer and the notifier, and a power supply configured to supply needed power to the measurer, the notifier, and the controller.

According to an example embodiment, the microneedle patch 101 may have a minimized size through a MEMS technology, and be configured as an active device using, as the power supply, a general battery, an ultrasmall charge and discharge battery, or an ultrasmall supercapacitor. In addition, in the case of a manual type of which a communicator of the microneedle patch 101 uses LC resonance formed with an inductor and a capacitor, an antenna capable of wirelessly communicating with the microneedle patch 101 of the manual type may need to be included in a communication terminal such as a mobile phone or a smartphone. When using the microneedle patch 101 of the manual type, the wireless communication between the microneedle patch 101 and the communication terminal may be performed by a magnetic induction-based coupling method. That is, using a principle of supplying power to the microneedle patch 101 using an electromotive force generated from the antenna of such an external terminal, it is possible to configure it as a circuit that does not have a separate power supply.

According to an example embodiment, the power supply may be used to operate a microcontroller, and supply power to the measurer that requires a separate power supply. In this case, the microcontroller may store a separate measurement process in a memory, and the measurement process may be performed according to a procedure programmed individually according to a type of data to be measured. An initial state may be a standby state in which power is not supplied to the microcontroller. When a wake-up signal is received from an external device, a measurement may be started in the standby state.

According to an example embodiment, the microneedle patch 101 may further include an amplifier configured to amplify a signal measured by the measurer and an ADC configured to digitize a signal amplified by the amplifier.

The signal measured by the measurer may be transmitted to the notifier through the communicator, and the notifier may be, for example, a mobile phone or a smartphone. The measured signal may be transmitted and received using a technology for wireless communication between the measurer and the mobile phone or smartphone. For example, in the case of a manual type of which the communicator uses LC resonance formed with an inductor and a capacitor, an antenna capable of wirelessly communicating with the measurer of the manual type may need to be embedded in a communication terminal such as the mobile phone or smartphone. When using the measurer of the manual type, the wireless communication between the measurer and the communication terminal may be performed by a magnetic induction-based coupling method. When transmitting and receiving data using the magnetic induction-based coupling method, the terminal may need to be disposed within an appropriate distance with the terminal and the measurer being disposed on a straight line. In addition, it is possible to transmit and receive data wirelessly by embedding a separate communication module for Zigbee communication and Bluetooth communication in the terminal and the measurer as a pair, and establish the wireless communication by providing a separate communication module outside the terminal. In this case, using the wireless communication module, it is possible to transmit and receive data within 10±5 m regardless of positions of the measurer and the terminal.

According to an example embodiment, all methods of measuring blood sugar using the microneedle patch 101 may be started by a content of a mobile phone or a terminal, or an application of a smartphone. The content or application may be individually configured to interwork with a certain patch, for example, the microneedle patch 101, and may also be configured as an integrated content or application capable of interworking with all patches including, for example, the microneedle patch 101. For example, when, in the content and application, a type of device for measurement, for example, the microneedle patch 101, is selected and personal information is input, and a start button is then pressed, the content or application may transmit a wake-up signal to the patch 101. By the wake-up signal, the measurement by the patch 101 may be started. The content or application may display a current state of the user by comparing, to existing reference data, a data value received through the communicator and other result values. In this case, when a health condition of the user is out of a normal range, for example, when the user has an abnormal concentration of sugar in the body, the content or application may provide the user with a simple health management method and provide, at the same time, information for the user to transmit related data to his/her guardian and doctor. However, when a signal is weak due to a poor attachment between a portion of the user being measured and the patch 101, or when a signal is weak due to a poor contact between the patch 101 and the terminal, the content or application may transmit such a problem to the user such that the measurement is performed smoothly.

According to an example embodiment, the notifier may visually provide the user with the color measured by the measurer through a display.

According to an example embodiment, the sensor 200 may include the polymer matrix 250 that reversibly expresses a different color based on a sugar concentration, and/or a chromogenic body 260 that reversibly expresses a different color by sensing electrons, hydrogen peroxide, or a change in pH that are generated by a reaction between the sugar and the enzyme 230.

Hereinafter, an example where the polymer matrix 250 that reversibly expresses a different color based on a concentration of sugar is included, and an example where the chromogenic body 260 that reversibly expresses a different color by sensing electrons, hydrogen peroxide, or a change in pH that are generated by a reaction between the sugar and the enzyme 230 will be described, respectively.

According to an example embodiment, the microneedle patch 101 including the polymer matrix 250 that reversibly expresses a different color based on a sugar concentration will be first described hereinafter with reference to FIG. 7.

According to an example embodiment, the polymer matrix 250 may be in contact with an upper side of the microneedle 220. That is, a blood fluid in the skin 110 of the human body may be extracted into the polymer matrix 250 through the microneedle 220, and sugar included in the extracted body fluid may directly react with the polymer matrix 250 and a color of the polymer matrix 250 may thus change based on a concentration of the sugar.

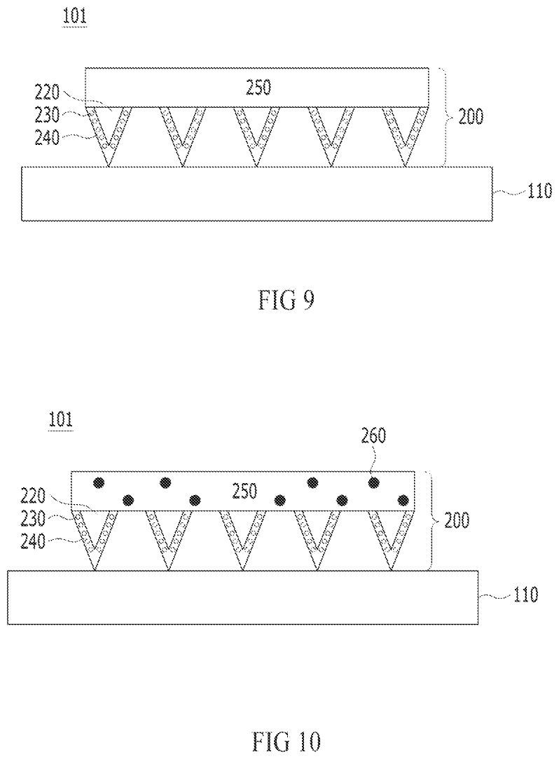

According to an example embodiment, the microneedle patch 101 including the chromogenic body 260 that reversibly expresses a different color by sensing electrons, hydrogen peroxide, or a change in pH that are generated by a reaction between the sugar and the enzyme 230 will be described with reference to FIGS. 9 through 12.

The sensor 200 may include the enzyme 230 that reacts with sugar included in the body fluid, and the enzyme 230 may be desirably glucose oxidase (GOx). In this case, the glucose oxidase (GOx) may react with the sugar, which is represented by Reaction Equation 1 above. The sugar described herein may also be referred to as glucose.

That is, as represented by Reaction Equation 1 above, the glucose oxidase may react with the sugar (or glucose) to generate hydrogen peroxide (H₂O₂) and/or electrons (e⁻). In this case, the glucose oxidase is oxidized again after being reduced, and may thus react reversibly with the sugar, enabling a continuous reaction. In addition, pH may change by the generated hydrogen peroxide. Thus, the enzyme 230 may represent the electrons, the hydrogen peroxide, or the pH change as it reacts with the sugar in the body fluid.

According to an example embodiment, the sensor 200 may include the chromogenic body 260 that reversibly expresses a different color by sensing electrons, hydrogen peroxide, or a change in pH that are generated by a reaction between the sugar in the body fluid and the enzyme 230.

The chromogenic body 260 may include a substance selected from a group consisting of a magnetic nanoparticle, a ruthenium complex, a chromogenic indicator, a dye, a para-hydroxyphenyl acetic acid, a lectin, and combinations thereof.

The magnetic nanoparticle may be peroxidase-active and include a substance selected from a group consisting of iron oxide, ferrite, alloy, and combinations thereof. In detail, the iron oxide may be, for example, Fe₂O₃ or Fe₃O₄. The ferrite may be, for example, CoFe₂O₄ or MnFe₂O₄. The alloy may be, for example, FePt or CoPt. However, the magnetic nanoparticle may not directly generate a color change by reacting with hydrogen peroxide, but generate the color change through a chromogenic substrate. In this case, the chromogenic substrate may include a substance selected from a group consisting of Amplex Red, ABTS, TMB, o-phenylenediamine dihydrochloride (OPD), 3,3′-diaminobenzidine (DAB), and combinations thereof. The magnetic nanoparticle may be effectively used as the chromogenic body 260 that reversibly expresses a color because it is effectively separable and reusable using a magnetic force.

The ruthenium complex may be ruthenium II complex (Ru(ddp)₃²), and used to measure a concentration of sugar using a characteristic that its fluorescence intensity decreases because oxygen consumption increases as the concentration of sugar increases.

The chromogenic indicator may react with the generated hydrogen peroxide in the presence of peroxidase to generate a different color or chemiluminescence, and include, for example, 3-hydroxy-2,4,6-triiodide benzoic acid or 3-hydroxy-2,4,6-tribromobenzoic acid.

The dye may be a xanthene-type dye or a fluorescein-type dye, and may generate an optical signal that changes dependently on a concentration of sugar based on the concentration of sugar.

The para-hydroxyphenyl acetic acid may exhibit strong fluorescence in proportion to a concentration of sugar, and additionally use a ruthenium porphyrin complex (RuO₂) as a catalyst for decomposing a dimer to achieve a reversible reaction.

The lectin may be a sugar-binding lectin, for example, Con A or glucose oxidase.

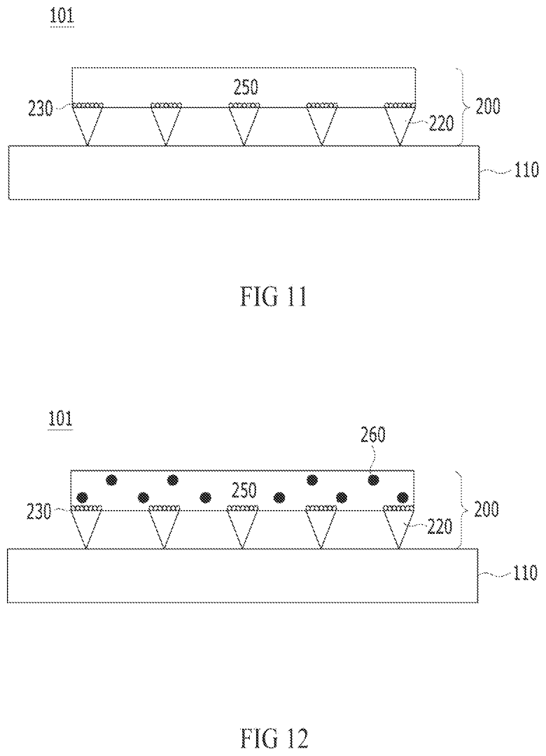

According to an example embodiment, the sensor 200 may include the polymer matrix 250 in contact with an upper side of the microneedle 220, and the chromogenic body 260 may be dispersed in the polymer matrix 250. In this case, the enzyme 230 may be dispersed in the microneedle 220 (refer to FIGS. 9 and 10) or be dispersed in the polymer matrix 250 (refer to FIGS. 11 and 12).

When the enzyme 230 is dispersed in the microneedle 220, the microneedle 220 may further include a coating layer 240 on an outer side thereof. In this case, the coating layer 240 may be of a porous structure including a plurality of pores through which other components of relatively large size included in the body fluid may not pass, but sugar of relatively small size may pass. In this case, sugar included in the body fluid may penetrate the coating layer 240 and react with the enzyme 230 dispersed in the microneedle 220 to generate electrons or hydrogen peroxide, or change pH thereby. The generated electrons, hydrogen peroxide, or pH change occurring thereby may react to the chromogenic body 260 dispersed in the polymer matrix 250.

That the enzyme 230 is dispersed in the polymer matrix 250 may be desirably a case in which the enzyme 230 is dispersed in a portion in contact with the microneedle 220 in the polymer matrix 250. In this case, sugar included in the body fluid may be extracted into the polymer matrix 250 through the microneedle 220, and the extracted sugar may react with the enzyme 230 dispersed in the polymer matrix 250. Thus, the electrons, hydrogen peroxide, or pH change may be generated or occur. The generated electrons, hydrogen peroxide, or pH change may react to the chromogenic body 260 dispersed in the polymer matrix 250, thereby expressing or developing a color.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

1. A realtime blood sugar monitoring device, comprising:
a sensor comprising a microneedle configured to invade a skin of a human body;
a measurer configured to measure a color generated by the sensor; and
a notifier configured to notify a user of the color measured by the measurer,
wherein the sensor is configured to express the color by reacting with sugar comprised in a body fluid in the skin of the human body.

2. The realtime blood sugar monitoring device of claim 1, wherein the sensor is configured to reversibly express a different color based on a concentration of the sugar.

3. The realtime blood sugar monitoring device of claim 2, wherein the sensor comprises a polymer matrix that reversibly expresses the different color based on the concentration of the sugar.

4. The realtime blood sugar monitoring device of claim 3, wherein the polymer matrix is in contact with an upper side of the microneedle.

5. The realtime blood sugar monitoring device of claim 2, wherein the sensor comprises an enzyme that reacts with the sugar comprised in the body fluid.

6. The realtime blood sugar monitoring device of claim 5, wherein the enzyme is glucose oxidase.

7. The realtime blood sugar monitoring device of claim 5, wherein the sensor comprises a polymer matrix in contact with an upper side of the microneedle.

8. The realtime blood sugar monitoring device of claim 5, wherein the sensor comprises a chromogenic body that reversibly expresses the different color by sensing an electron, hydrogen peroxide, or a change in pH that are generated from the reaction between the sugar comprised in the body fluid and the enzyme.

9. (canceled)

10. The realtime blood sugar monitoring device of claim 1, configured to release, to an outside, the body fluid that is already reacted on a periodic basis.

11. The realtime blood sugar monitoring device of claim 1, further comprising:
a controller configured to control the measurer and the notifier; and
a power supply configured to supply needed power to the measurer, the notifier, and the controller.

12. The realtime blood sugar monitoring device of claim 11, further comprising:
an amplifier configured to amplify a signal measured by the measurer; and
an analog-to-digital converter (ADC) configured to digitize a signal amplified by the amplifier.

13. The realtime blood sugar monitoring device of claim 1, wherein the notifier is configured to convert the color measured by the measurer into the concentration of the sugar in the body fluid and visually provide the user with a result of the converting through a display.

14. (canceled)

15. A microneedle patch, comprising:
a microneedle configured to invade a skin of a human body; and
a sensor configured to express a color by coming into contact with the microneedle and reacting with sugar comprised in a body fluid in the skin of the human body.

16. The microneedle patch of claim 15, wherein the sensor is configured to reversibly express a different color based on a concentration of the sugar.

17. The microneedle patch of claim 16, wherein the sensor comprises a polymer matrix that reversibly expresses the different color based on the concentration of the sugar.

18. The microneedle patch of claim 17, wherein the polymer matrix is in contact with an upper side of the microneedle.

19. The microneedle patch of claim 16, wherein the sensor comprises an enzyme that reacts with the sugar comprised in the body fluid.

20. The microneedle patch of claim 19, wherein the enzyme is glucose oxidase.

21. The microneedle patch of claim 19, wherein the sensor comprises a polymer matrix in contact with an upper side of the microneedle.

22. The microneedle patch of claim 19, wherein the sensor comprises a chromogenic body that reversibly expresses the different color by sensing an electron, hydrogen peroxide, or a change in pH that are generated from the reaction between the sugar comprised in the body fluid and the enzyme.

23. (canceled)
